(19) United States
(12) Patent Application Publication
(10) Pub. No.: US 2010/0018528 A1
(43) Pub. Date: Jan. 28, 2010

(54) BOTTLE CLOSURE WITH SELF-SEALING MEMBRANE

(76) Inventor: Michael CUZYDLO, Orchard Park, NY (US)

Correspondence Address: REED SMITH LLP, P.O. BOX 488, PITTSBURGH, PA, 15230-0488, US

(21) Appl. No.: 12/507,420

(22) Filed: Jul. 22, 2009

Related U.S. Application Data
(60) Provisional application No. 61/082,596, filed on Jul. 22, 2008.

(51) Int. Cl. A61M 16/01

(52) U.S. Cl. 128/203.21

(57) ABSTRACT

An apparatus, system and method for transferring a liquid, such as a liquid anesthetic, from a reservoir to a machine while minimizing the release of a liquid to the surrounding environment. The apparatus may include a device having a reservoir; and a self-sealing membrane attached to the reservoir. The system may include a device having a reservoir and a self-sealing membrane attached to the reservoir; and a vaporizer having a needle for extending through the self-sealing membrane when the device is mated with the vaporizer.

### CROSS-REFERENCE TO RELATED APPLICATION

This application claims the benefit under 35 U.S.C. §119(e) of the earlier filing date of U.S. Provisional Application Ser. No. 61/082,596 filed on Jul. 22, 2008, the entire disclosure of which is hereby incorporated by reference herein as if being set forth in its entirety.

### FIELD OF THE INVENTION

This application discloses an invention which is related, generally and in various embodiments, to a device, system and method for supplying a liquid to a machine, such as a vaporizer.

### BACKGROUND OF THE INVENTION

Liquid anesthetics are often packaged in glass bottles and shipped to a location where they may be used to anesthetize a patient undergoing a medical or dental procedure. Such anesthetics may also be used to induce analgesia in a patient undergoing a medical or dental procedure. In order to administer the anesthetic, the contents of the glass bottle are placed in a vaporizer. The vaporizer can vaporize the anesthetic and provide the vaporized anesthetic in a desired amount to the patient.

Inhalable anesthetics are typically volatile substances with relatively low boiling points and high vapor pressure. Preferably, there is little or no release of anesthetic to the atmosphere during handing. To transfer liquid anesthetic to a vaporizer, however, the bottle containing the vaporizer must be opened. Since it is unwise to expose medical personnel performing a procedure to an anesthetic, and since anesthetics are expensive, devices have been developed to minimize the release of anesthetic from a bottle to the environment surrounding a vaporizer. These devices, however, have failed to effectively minimize the release of anesthetic.

### SUMMARY OF THE INVENTION

This application discloses an apparatus, system and method for transferring a liquid, such as a liquid anesthetic, from a reservoir to a machine while minimizing the release of a liquid to the surrounding environment. The apparatus may include a device having a reservoir; and a self-sealing membrane attached to the reservoir. The system may include a device having a reservoir and a self-sealing membrane attached to the reservoir; and a vaporizer having a needle for extending through the self-sealing membrane when the device is mated with the vaporizer. The container may include a nozzle having a threaded connection to the vaporizer. The container may also include a cap attachable to the nozzle when the container is not connected to the vaporizer.

### BRIEF DESCRIPTION OF THE DRAWINGS

FIG. 1 illustrates an anesthetic agent container according to the invention;

FIG. 2 is a cross-section of the container in FIG. 1 taken along the line A-A in FIG. 1;

FIG. 3 illustrates an exploded view of the container depicted in FIG. 1; and

FIG. 4 illustrates a device according to the invention mated to a vaporizer.

### DETAILED DESCRIPTION OF THE INVENTION

The accompanying drawings are intended to provide further understanding of the invention and are incorporated in and constitute a part of the description of the invention. The drawings illustrate an embodiment of the invention and together with the description illustrate principles of the invention.

The drawings should not be taken as implying any necessary limitation on the essential scope of invention. The drawings are given by way of non-limitative example to explain the nature of the invention.

For a more complete understanding of the instant invention reference is now made to the following description taken in conjunction with accompanying drawings.

The various features of novelty which characterize the invention are pointed out specifically in the claims which are a part of this description. For a better understanding of the invention, reference should be made to the drawings and descriptive matter in which there are illustrated and described preferred embodiments of invention.

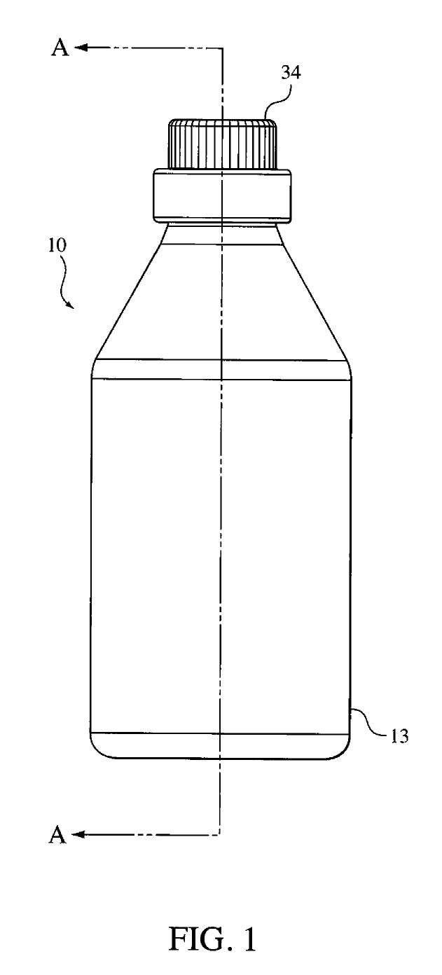

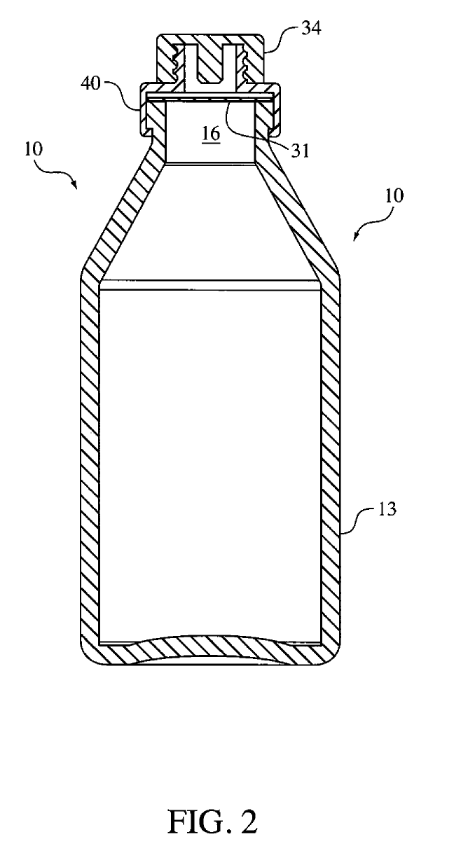

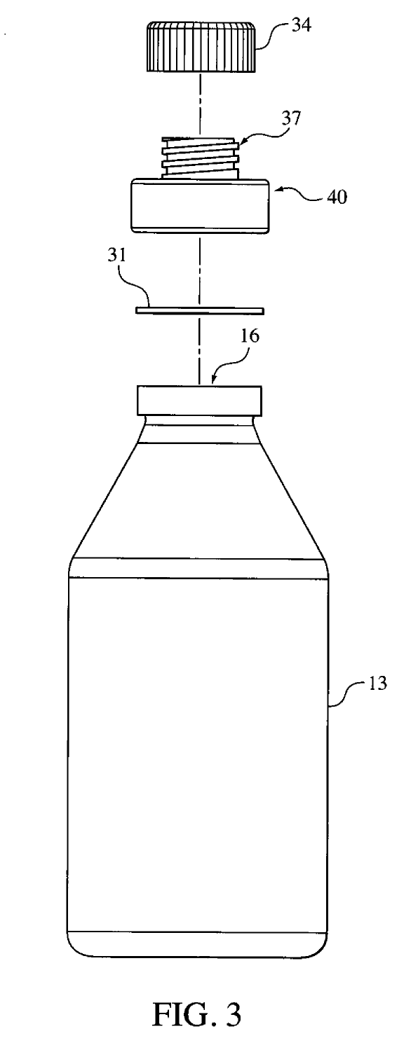

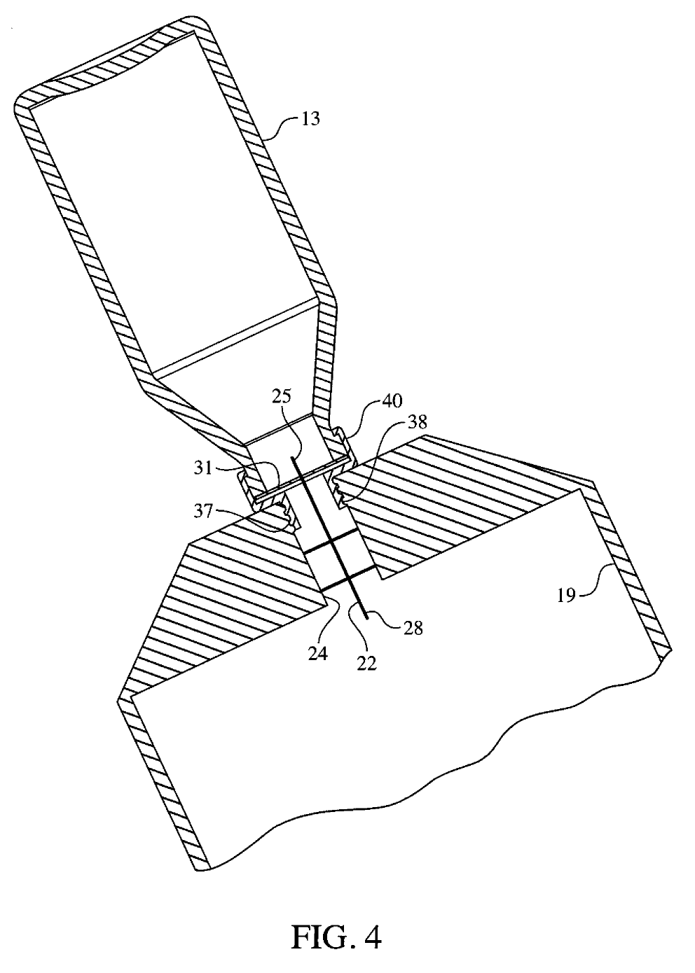

Referring now to the drawings, wherein like numerals designate identical or corresponding parts throughout the referred views, FIGS. 1-4 show various embodiments of an apparatus connecting a device 10 to a machine 19. The device 10 may include a reservoir 13, such as a glass bottle capable of holding a liquid such as a volatile anesthetic. At one end, the reservoir 13 includes an opening 16 (FIGS. 203) through which anesthetic may be transferred from the reservoir 13 to the machine 19 when it is desired to transfer the anesthetic from the reservoir 13. In this embodiment, the machine is a vaporizer that dispenses the anesthetic to a person undergoing a medical procedure. The vaporizer 19 may be equipped with a needle 22 secured within an opening 24 of the vaporizer. The needle 22 is adapted to carry anesthetic agent from a first end 25 to a second end 28.

The device 10 may include a self-sealing membrane 31 for selectively allowing the volatile anesthetic to leave the reservoir 13. The membrane 31 may be made from a rubbery material such as FDA grade ethylene propylene diene M-class “EPDM” rubber. When the device 10 is inserted into the opening 24 of the vaporizer 19, the needle 22 pierces and extends through the membrane 31 so that the first end 25 is inside the reservoir 13. In this position, the anesthetic flows through the needle 22 from the first end 25 to the second end 28, and in so doing the anesthetic flows by gravity from the reservoir 13 to the vaporizer 19.

Once a desired amount of the anesthetic has been emptied from the reservoir 13 to the vaporizer 19, the device 10 may be removed from the vaporizer 19. As the device 10 is removed, the membrane 31 is moved away from the needle 22. When the needle 22 no longer extends through the membrane 31, the self-sealing nature of the membrane 31 closes the hole through which the needle 22 previously extended. In this manner, any anesthetic remaining in the reservoir 13 is prevented from escaping from the reservoir 13.

A cap 34 may be provided to protect the membrane 31 when it is desired that the anesthetic should remain in the reservoir 13, for example during shipping of the device 10. The cap 34 may have threads, which may be used to engage a threaded surface 37 provided by a nozzle 40. The threads 37 also may be used to engage a threaded surface 38 of the opening 24 of the vaporizer 19, and in that manner hold the reservoir 13 to the vaporizer 19 while the anesthetic is transferred from the reservoir 13 to the vaporizer 19. The nozzle 40 may be attached to the reservoir 13 by crimping the nozzle to the reservoir 13.

It is to be understood that the descriptions of the invention have been simplified to illustrate characteristics that are relevant for a clear understanding of the invention. Those of ordinary skill in the art may recognize that other elements or steps are desirable or required in implementing the invention. However, because such elements or steps are well known in the art, and because they do not facilitate a better understanding of the invention, a discussion of such elements or steps is not provided herein. The disclosure herein is directed to all such variations and modifications to such elements and methods known to those skilled in the art.

It is to be understood that the invention may assume various alternative orientations and step sequences, except where expressly specified to the contrary. It is also to be understood that the specific devices and processes illustrated in the attached drawings, and described in this specification are simply exemplary embodiments of the inventive concepts defined in the appended claims. Hence, specific dimensions and other physical characteristics relating to the embodiments disclosed herein are not to be considered as limiting, unless the claims expressly state otherwise.

Further, since numerous modifications and changes will readily occur to those skilled in the art, it is not desired to limit the invention to the exact construction and operation shown and described, and accordingly all suitable modifications and equivalents may be regarded as falling within the scope of the invention as defined by the claims that follow.

What is claimed is:

1. An anesthetic container for transferring liquid anesthesia to a vaporizer, the container comprising:
a reservoir adapted for containing the liquid anesthesia, an opening at one end of the reservoir; and
a self-sealing membrane attached to the reservoir and sealing the opening, wherein the membrane is pierceable by a needle for removal of liquid anesthetic from the container for transfer to the vaporizer, and wherein the membrane is resealable after withdrawal of the needle from the membrane.

2. The container of claim 1, wherein the self-sealing membrane comprises ethylene propylene diene M-class rubber.

3. The container of claim 1, wherein the container is connectable to a vaporizer.

4. The container of claim 1, wherein the container further comprises a nozzle attached to the reservoir adapted for connection to a vaporizer.

5. The container of claim 4, wherein the nozzle defines threads adapted for connection to a threaded opening in the vaporizer.

6. The container of claim 5, wherein the container further includes a cap connectable to the nozzle threads when the container is not connected to a vaporizer.

7. An anesthetic agent delivery system comprising:
a container having a reservoir having an opening at one end and a self-sealing membrane attached to the reservoir sealing the opening to the reservoir; and
a vaporizer having a needle for extending through the self-sealing membrane when the container is connected to the vaporizer;
wherein the membrane is pierceable by the needle for removal of liquid anesthetic from the container for transfer to the vaporizer, and wherein the membrane is resealable after withdrawal of the needle from the membrane.

8. The anesthetic agent delivery system of claim 7, wherein the self-sealing membrane comprises ethylene propylene diene M-class rubber.

9. The anesthetic agent delivery system of claim 7, wherein the container is connectable to the vaporizer.

10. The anesthetic agent delivery system of claim 7, wherein the container further comprises a nozzle attached to the reservoir for connection to the vaporizer.

11. The anesthetic agent delivery system of claim 10, wherein the nozzle defines threads for connection to a threaded opening in the vaporizer.

12. The anesthetic agent delivery system of claim 11, wherein the container further includes a cap connectable to the nozzle threads when the container is not connected to the vaporizer.

13. A method of transferring liquid anesthesia from a container to a vaporizer, the method comprising:
connecting a container having a reservoir which holds a liquid anesthetic to a vaporizer;
piercing a self-sealing membrane sealing an opening of the reservoir with a needle attached to the vaporizer, wherein liquid anesthesia flows through the needle from the container into the vaporizer.

14. The method of claim 13, wherein piercing the membrane occurs while connecting the container to the vaporizer.

15. The method of claim 13, wherein connecting the container comprises threadedly connecting the container to the vaporizer.

16. The method of claim 13, further comprising removing the container from the vaporizer upon completion of transferring liquid anesthesia from the container to the vaporizer.

17. The method of claim 16, wherein removing the container from the vaporizer includes withdrawing the needle from the membrane, and wherein the membrane reseals after withdrawal of the needle from the membrane.
